(19) United States
(12) Patent Application Publication
(10) Pub. No.: US 2024/0065558 A1
(43) Pub. Date: Feb. 29, 2024

(54) Patient Identification And Vitals Screening Device

(71) Applicant: Ebony Wright, Monticello, NY (US)

(72) Inventor: Ebony Wright, Monticello, NY (US)

(21) Appl. No.: 17/895,745

(22) Filed: Aug. 25, 2022

(51) Int. Cl. A61B 5/0205; A61B 5/00; A61B 5/024; A61B 5/1172; G16H 10/60

(52) U.S. Cl. CPC A61B 5/0205; A61B 5/0022; A61B 5/02433; A61B 5/1172; A61B 5/6887; A61B 5/7405; G16H 10/60; A61B 2505/07; A61B 2560/0214

(57) ABSTRACT

A patient identification and vitals screening device for rapid identification and vitals screening of a patient includes a fingerprint scanner and a pulse oximeter, which are attached to a housing that defines an interior space. The fingerprint scanner generates a fingerprint scan from a finger of a patient that is positioned upon the fingerprint scanner so that the patient can be identified. The pulse oximeter is positioned within the fingerprint scanner and measures a pulse rate and a blood oxygen level of the patient. The patient identification and vitals screening device can be engaged to an electronic device that is in possession of a healthcare worker. The electronic device is engaged to a server upon which a plurality of fingerprint scans and a plurality of healthcare records is positioned. The server identifies the patient and presents their healthcare record to the healthcare worker.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to identification devices and more particularly pertains to a new identification device for rapid identification and vitals screening of a patient. The present invention discloses an identification device comprising a fingerprint scanner and a pulse oximeter.

### (2) Description of Related Art Including Information Disclosed Under 37 CFR 1.97 and 1.98

The prior art relates to identification devices, which may comprise combination fingerprint scanners and pulse readers used in systems for purchasing and financial transactions and for identification. The prior art also biometric scanners used in reservation systems and finger probes used to measure blood oxygenation. What is lacking in the prior art is an identification device comprising a fingerprint scanner and a pulse oximeter that enable rapid identification and vitals screening of a patient. Additionally lacking in the prior art is a system incorporating such an identification device wherein healthcare records of the patient can be displayed to a healthcare worker upon verification of the fingerprint scan.

### BRIEF SUMMARY OF THE INVENTION

An embodiment of the disclosure meets the needs presented above by generally comprising a fingerprint scanner and a pulse oximeter, which are attached to a housing that defines an interior space. The fingerprint scanner is configured to generate a fingerprint scan from a finger of a patient that is positioned upon the fingerprint scanner so that the patient can be identified. The pulse oximeter is positioned within the fingerprint scanner and is configured to measure a pulse rate and a blood oxygen level of the patient.

Another embodiment of the disclosure includes a patient identification and vitals screening system, which comprises a patient identification and vitals screening device 10, a server, and an electronic device in possession of, or accessible to, a healthcare worker. A plurality of fingerprint scans and a plurality of healthcare records are positioned on the server, with each healthcare record being associated with a respective fingerprint scan. The electronic device is communicatively engaged to the server. The server is enabled to identify the patient by comparison of the fingerprint scan obtained by the fingerprint scanner to the plurality of fingerprint scans and to present an associated healthcare record on the electronic device of the healthcare worker.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a top view of a patient identification and vitals screening device according to an embodiment of the disclosure.

FIG. 2 is a bottom view of an embodiment of the disclosure.

FIG. 3 is a block diagram of an embodiment of the disclosure.

FIG. 4 is an in-use view of an embodiment of the disclosure.

### DETAILED DESCRIPTION OF THE INVENTION

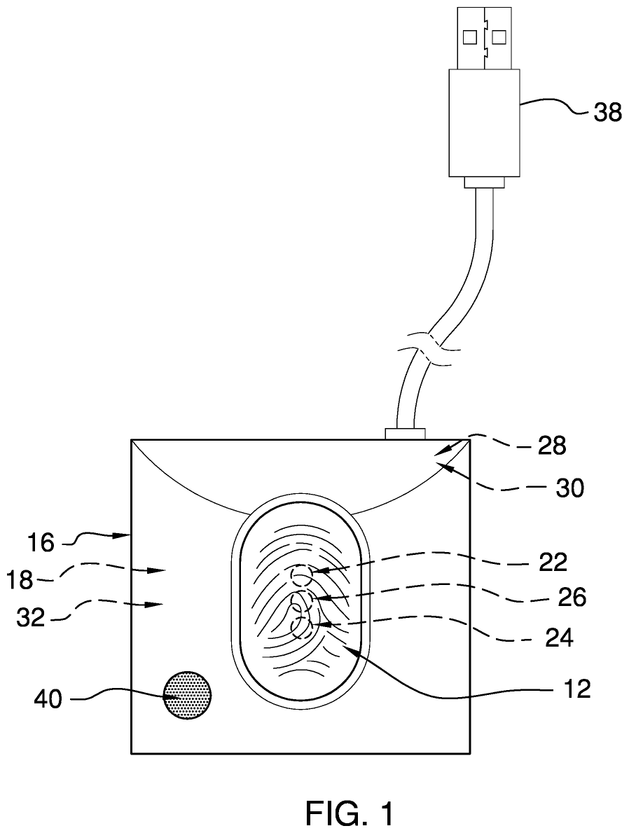

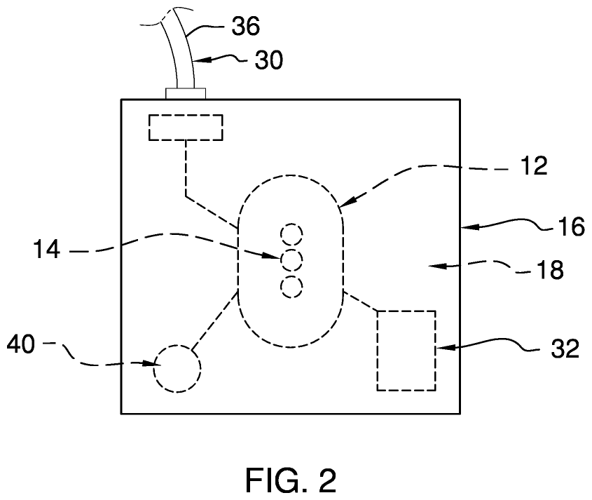

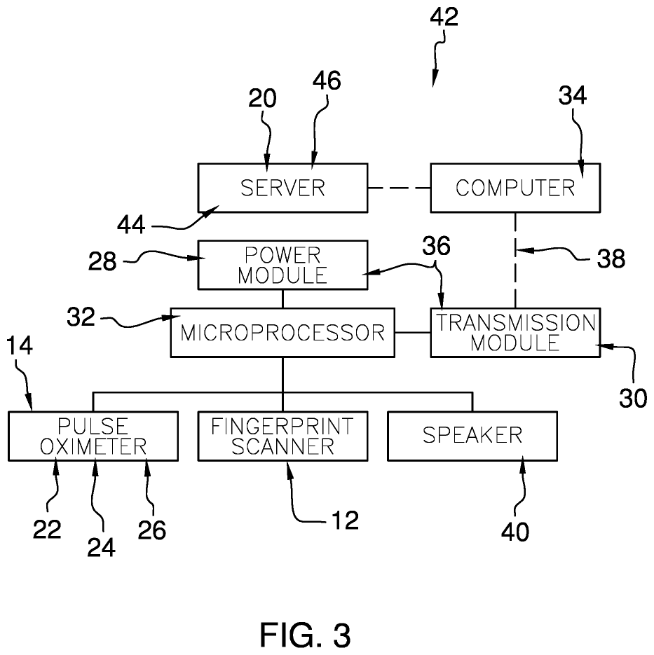

With reference now to the drawings, and in particular to FIGS. 1 through 4 thereof, a new identification device embodying the principles and concepts of an embodiment of the disclosure and generally designated by the reference numeral 10 will be described.

As best illustrated in FIGS. 1 through 4, the patient identification and vitals screening device 10 generally comprises a fingerprint scanner 12 and a pulse oximeter 14, which are attached to a housing 16 that defines an interior space 18. The fingerprint scanner 12 is configured to generate a fingerprint scan 20 from a finger of a patient that is positioned upon the fingerprint scanner 12 so that the patient can be identified. The pulse oximeter 14 is positioned within the fingerprint scanner 12 and is configured to measure a pulse rate and a blood oxygen level of the patient.

Scanners for fingerprints are known to those skilled in the art of biometric scanners and may comprise optical scanners, capacitive scanners, ultrasonic scanners, thermal scanners, and the like. The pulse oximeter 14 comprises a red light emitting diode 22, an infrared light emitting diode 24, and a photodiode 26, which is positioned to detect red light and infrared light that is reflected from tissues of the finger positioned upon the fingerprint scanner 12. The present invention also anticipates the housing 16 being configured such that the photodiode 26 is positioned to detect transmitted light. Such a configuration of the housing 16 might resemble clip-on pulse oximetry devices found in the prior art, wherein the photodiode 26 is opposing to the red light emitting diode 22 and the infrared light emitting diode 24.

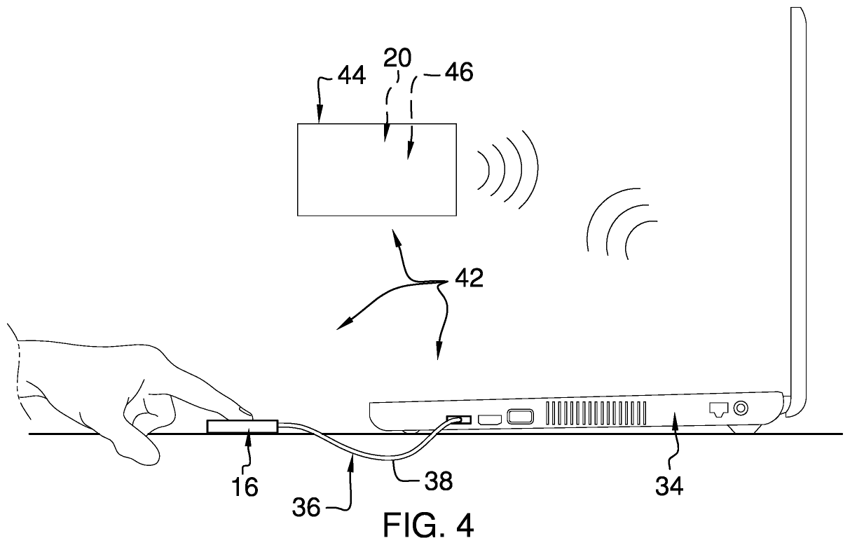

A power module 28, a transmission module 30, and a microprocessor 32 are attached to the housing 16 and are positioned in the interior space 18. The microprocessor 32 is operationally engaged to the fingerprint scanner 12, the pulse oximeter 14, the power module 28, and the transmission module 30. The microprocessor 32 thus is enabled to selectively actuate the transmission module 30 to transmit the fingerprint scan 20, the pulse rate, and the blood oxygen level of the patient to an electronic device 34 of a healthcare worker. The electronic device 34 could comprise a desktop computer, laptop computer (as shown in FIG. 4), table computer, smartphone, or the like.

The transmission module 30 and the power module 28 may comprise a data and power cable 36, such as, but not limited to, a Universal Serial Bus cable 38, as shown in FIG. 1. The present invention also anticipates the transmission module 30 and the power module 28 comprising a transmitter and a battery, respectively, thus enabling cordless operation of the patient identification and vitals screening device 10.

The patient identification and vitals screening device 10 also may comprise a speaker 40, which is attached to the housing 16 and which is operationally engaged to the microprocessor 32. The microprocessor 32 is enabled to selectively actuate the speaker 40 to broadcast an audio signal, such as a beep or chime, upon the fingerprint scanner 12 obtaining the fingerprint scan 20, thus letting the patient know that they can remove their finger from the fingerprint scanner 12.

Another embodiment of the disclosure includes a patient identification and vitals screening system 42, which comprises a patient identification and vitals screening device 10, a server 44, and the electronic device 34 that is in possession of, or accessible to, the healthcare worker. A plurality of fingerprint scans 20 and a plurality of healthcare records 46 are positioned on the server 44, with each healthcare record 46 being associated with a respective fingerprint scan 20.

The electronic device 34 is communicatively engaged to the server 44. The server 44 is enabled to identify the patient by comparison of the fingerprint scan 20 that is obtained by the fingerprint scanner 12 to the plurality of fingerprint scans 20. The server 44 then can selectively present an associated healthcare record 46 on the electronic device 34 of the healthcare worker. The present invention is anticipated to be useful in speeding up check-in processes at healthcare facilities, thus shortening the time between patient arrival and their interaction with a medical professional.

In use, the patient positions their finger upon fingerprint scanner 12, which generates the fingerprint scan 20 so that the patient can be identified. Concurrently, the pulse oximeter 14 measures the pulse rate and the blood oxygen level of the patient. The server 44 identifies the patient and presents their healthcare record 46 on the electronic device 34 of the healthcare worker for their review.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A patient identification and vitals screening device comprising:
a housing defining an interior space;
a fingerprint scanner attached to the housing, wherein the fingerprint scanner is configured for generating a fingerprint scan from a finger of a patient positioned upon the fingerprint scanner, such that the patient can be identified; and
a pulse oximeter attached to the housing and positioned within the fingerprint scanner, wherein the pulse oximeter is configured for measuring a pulse rate and a blood oxygen level of the patient.

2. The patient identification and vitals screening device of claim 1, wherein the pulse oximeter comprises:
a red light emitting diode;
an infrared light emitting diode; and
a photodiode positioned for detecting red light and infrared light reflected from tissues of the finger positioned upon the fingerprint scanner.

3. The patient identification and vitals screening device of claim 1, further including:
a power module attached to the housing and positioned in the interior space;
a transmission module attached to the housing and positioned in the interior space; and
a microprocessor attached to the housing and positioned in the interior space, the microprocessor being operationally engaged to the fingerprint scanner, the pulse oximeter, the power module, and the transmission module, such that the microprocessor is enabled for selectively actuating the transmission module for transmitting the fingerprint scan, the pulse rate, and the blood oxygen level of the patient to an electronic device of a healthcare worker.

4. The patient identification and vitals screening device of claim 3, wherein the transmission module and the power module comprise a data and power cable.

5. The patient identification and vitals screening device of claim 4, wherein the data and power cable comprising a Universal Serial Bus cable.

6. The patient identification and vitals screening device of claim 3, further including:
a speaker attached to the housing; and
the microprocessor being operationally engaged to the speaker, such that the microprocessor is enabled for selectively actuating the speaker for broadcasting an audio signal upon the fingerprint scanner obtaining the fingerprint scan.

7. A patient identification and vitals screening device comprising:
a housing defining an interior space;
a fingerprint scanner attached to the housing, wherein the fingerprint scanner is configured for generating a fingerprint scan from a finger of a patient positioned upon the fingerprint scanner, such that the patient can be identified;
a pulse oximeter attached to the housing and positioned within the fingerprint scanner, wherein the pulse oximeter is configured for measuring a pulse rate and a blood oxygen level of the patient, the pulse oximeter comprising:
a red light emitting diode,
an infrared light emitting diode, and
a photodiode positioned for detecting red light and infrared light reflected from tissues of the finger positioned upon the fingerprint scanner;
a speaker attached to the housing;
a power module attached to the housing and positioned in the interior space;
a transmission module attached to the housing and positioned in the interior space, the transmission module and the power module comprising a data and power cable, the data and power cable comprising a Universal Serial Bus cable; and
a microprocessor attached to the housing and positioned in the interior space, the microprocessor being operationally engaged to the fingerprint scanner, the pulse oximeter, the power module, and the transmission module, such that the microprocessor is enabled for selectively actuating the transmission module for transmitting the fingerprint scan, the pulse rate, and the blood oxygen level of the patient to an electronic device of a healthcare worker, the microprocessor being operationally engaged to the speaker, such that the microprocessor is enabled for selectively actuating the speaker for broadcasting an audio signal upon the fingerprint scanner obtaining the fingerprint scan.

8. A patient identification and vitals screening system comprising:
a server;
a plurality of fingerprint scans positioned on the server;
a plurality of healthcare records positioned on the server, each healthcare record being associated with a respective fingerprint scan;
an electronic device in possession of, or accessible to, a healthcare worker, the electronic device being communicatively engaged to the server;
a patient identification and vitals screening device operationally engaged to the electronic device and comprising:
a housing defining an interior space,
a fingerprint scanner attached to the housing, wherein the fingerprint scanner is configured for generating a fingerprint scan from a finger of a patient positioned upon the fingerprint scanner
a pulse oximeter attached to the housing and positioned within the fingerprint scanner, wherein the pulse oximeter is configured for measuring a pulse rate and a blood oxygen level of the patient,
a power module attached to the housing and positioned in the interior space;
a transmission module attached to the housing and positioned in the interior space, and
a microprocessor attached to the housing and positioned in the interior space, the microprocessor being operationally engaged to the fingerprint scanner, the pulse oximeter, the power module, and the transmission module, such that the microprocessor is enabled for selectively actuating the transmission module for transmitting the fingerprint scan, the pulse rate, and the blood oxygen level of the patient to the electronic device of the healthcare worker; and
wherein the server is enabled for identifying the patient by comparison of the fingerprint scan obtained by the fingerprint scanner to the plurality of fingerprint scans and for presenting an associated healthcare record on the electronic device of the healthcare worker.
